(19) United States
(12) Patent Application Publication
(10) Pub. No.: US 2008/0172012 A1
(43) Pub. Date: Jul. 17, 2008

(54) INJECTION NEEDLE HAVING LATERAL DELIVERY PORTS AND METHOD FOR THE MANUFACTURE THEREOF

(76) Inventors: Prasanga D. Hiniduma-Lokuge, Minneapolis, MN (US); Daniel C. Sigg, Saint Paul, MN (US); John L. Sommer, Coon Rapids, MN (US); Matthew D. Bonner, Plymouth, MN (US); Brian C.A. Fernandes, Roseville, MN (US)

Correspondence Address: MEDTRONIC, INC., 710 MEDTRONIC PARKWAY NE, MINNEAPOLIS, MN, 55432-9924, US

(21) Appl. No.: 11/555,086

(22) Filed: Oct. 31, 2006

(51) Int. Cl. A61M 5/32

(52) U.S. Cl. 604/272

(57) ABSTRACT

An injection needle comprises an elongated body having an outer surface and an inner surface defining a longitudinal channel through the tubular body. The elongated body further comprises a distal end and at least one lateral delivery port extending from the inner surface to the outer surface proximate the distal end and fluidly coupled to the longitudinal channel. A distal tip is coupled to the distal end and comprises a radio-opaque material.

### TECHNICAL FIELD

This invention relates generally to a medical device and, more particularly, to an injection needle having lateral delivery ports and a method for the production thereof.

### BACKGROUND OF THE INVENTION

Syringes equipped with injection needles are commonly employed to introduce liquid medicine, or injectate, into patients' bodies. A typical syringe comprises a tubular barrel (e.g., plastic) having a plunger slidably coupled to its proximal end. The barrel's distal end includes a small aperture therethrough. An injection needle (e.g., metal) is attached (e.g., threadably, integrally, etc.) to the barrel's distal end. The needle comprises an elongated body (e.g., metal) having a longitudinal injectate channel therethrough, which is placed in fluid communication with the aperture when the needle is attached to the barrel. The distal tip of the needle has a bore therethrough and typically includes a bevel (e.g., standard bevel, short bevel, true short bevel, etc.) to form a sharp, pointed tip. To administer the injection, the needle's distal tip is utilized to pierce the tegument (e.g., skin) covering the injection site. The plunger is then depressed, and injectate held within the barrel is forced through the needle and into the injection site.

More recently, injection needles have been deployed on tissue injection catheters, which may be navigated through a patient's vasculature to an internal injection site not easily accessible from the patient's exterior. Tissue injection catheters are especially useful for administering local injections to tissue and organs (e.g., a local intramyocardial injection to a patient's heart) of injectates including, but not limited to, human cells (e.g., stem cells, adult primary cells, bone marrow derived cells, human dermal fibroblasts, blood derived cells, cord blood derived cells, adipose tissue derived cells, etc.), genetically transformed cells, proteins (e.g., growth factors, cytokines, chemokines, extra-cellular matrix proteins, etc.), plasma, autologous derived serum, genes, plasmids, siRNA, hydrogels (synthetic or natural), pharmacological agents, and various combinations thereof. A representative tissue injection catheter comprises an elongated flexible catheter having a retractable needle deployed at its distal end. A fixation helix and/or electrode are also optionally deployed proximate the catheter's distal end. After the distal end of the catheter is guided to an injection site, such as the atrium of the heart, the injection needle is extended, and the injectate is administered. The catheter may be equipped with a radio-opaque marker visible under fluoroscopy to assist in guiding the needle to the desired site.

Regardless of the type of medical device with which they are utilized, standard injection needles of the type described are limited in several respects. For example, the distal tip of a standard injection needle tends to core (rather than pierce) tissue during needle insertion into the tissue. Coring tissue increases tissue trauma and may result in blockage of the injectate channel of the needle. In addition, a standard injection needle provides a relatively limited zone of injectate dispersal, and thus exposes less tissue to the injectate when a subcutaneous or intramuscular injection is administered. Furthermore, in the event of tissue perforation (i.e., the passage of the needle's distal tip through the targeted tissue), a standard injection needle may deliver some portion of the injectate to the surrounding area and not to the injection site, which may decrease the therapeutic effectiveness of the injection. Tissue perforation is especially likely when a catheter-delivered needle administers an intramuscular injection to an injection site (e.g., an atrium of the heart) characterized by relatively thin tissue. As yet another limitation, standard injection needles cannot easily carry radio-opaque markers visible under fluoroscopy, which aid in the tracking of a catheter-delivered needle as described above.

Considering the foregoing, it should be appreciated that it would be desirable to provide an injection needle that may be utilized with a medical device (e.g., syringe, a tissue injection catheter, or other needle-carrying medical device) and that overcomes the limitations associated with standard injection needles; i.e., that resists coring tissue, that provides a relatively broad injectate dispersal zone, that decreases the likelihood that injectate will be lost as a result of tissue perforation, and that may be conveniently provided with a radio-opaque marker. It should further be appreciated that it would be desirable to provide a method for producing such a needle. Other desirable features and characteristics of the present invention will become apparent from the subsequent detailed description of the invention and the appended claims, taken in conjunction with the accompanying drawings and this background of the invention.

### BRIEF DESCRIPTION OF THE DRAWINGS

The following drawings are illustrative of particular embodiments of the invention and therefore do not limit the scope of the invention, but are presented to assist in providing a proper understanding. The drawings are not to scale (unless so stated) and are intended for use in conjunction with the explanations in the following detailed descriptions. The present invention will hereinafter be described in conjunction with the appended drawings, wherein like reference numerals denote like elements, and:

FIGS. 1 and 2 are isometric and cross-sectional views, respectively, of an injection needle including a plurality of lateral delivery ports in accordance with a first exemplary embodiment of the present invention;

FIG. 3 is a plan view of the injection needle shown in FIGS. 1 and 2 administering injectate to an atrial appendage after tissue perforation;

FIG. 4 is a flowchart illustrating a process for producing the needle shown in FIGS. 1-3 and other embodiments of the inventive injection needle;

FIG. 5 is an isometric view of a pre-formed tip that may be attached to the selected tubing when producing an embodiment of the injection needle in accordance with the process outlined in FIG. 4; and

FIGS. 6 and 7 are isometric views of second and third exemplary embodiments, respectively, of the inventive injection needle.

### DETAILED DESCRIPTION OF EXEMPLARY EMBODIMENTS

The following description is exemplary in nature and is not intended to limit the scope, applicability, or configuration of the invention in any way. Rather, the following description provides a convenient illustration for implementing various exemplary embodiments of the present invention. Various changes to the described embodiments may be made in either the function or the arrangement of the elements described herein without departing from the scope of the invention.

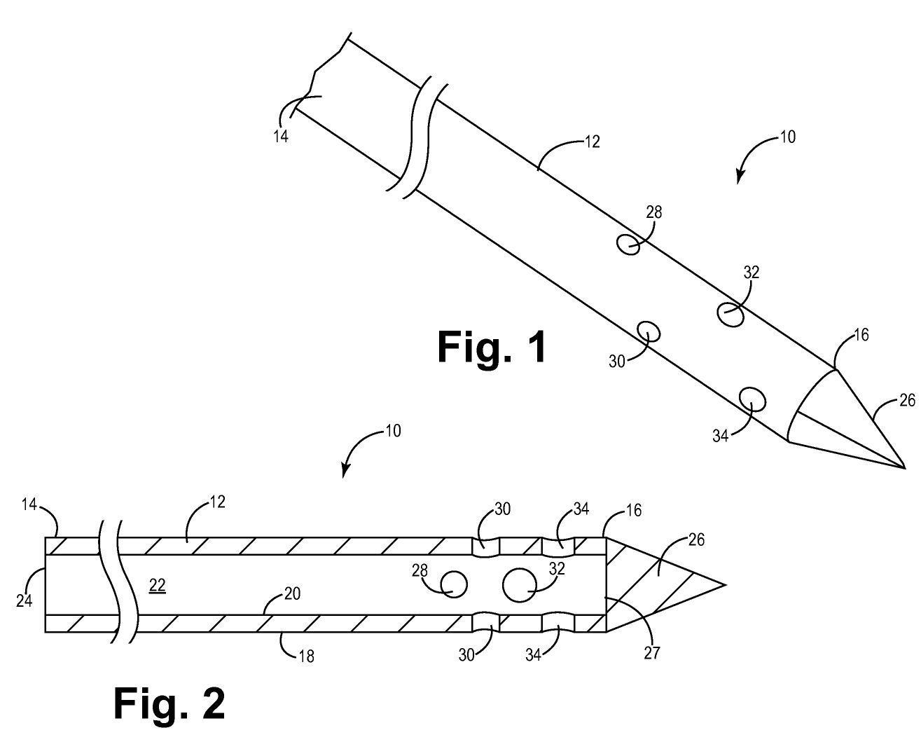

FIGS. 1 and 2 are isometric and cross-sectional views, respectively, of an injection needle 10 comprising an elongated body 12 having a proximal end 14 and a distal end 16. Elongated body 12 is substantially tubular and includes an outer surface 18 and an inner surface 20, which defines a longitudinal injectate channel 22 (FIG. 2) through body 12 from proximal end 14 to distal end 16. Proximal end 14 may be coupled to the distal end of a medical device (e.g., a syringe or a tissue injection catheter) in the well-known manner. An opening 24 (FIG. 2) is provided through proximal end 14 and permits longitudinal injectate channel 22 to receive a liquid injectate. The dimensions of elongated body 12 will vary depending upon application and needle gauge. If, for example, injection needle 10 is chosen to have a Stubs Needle Gauge of 27, the outer diameter of elongated body 12 may be approximately 0.014 inch, the inner diameter of body 12 (i.e., the outer diameter of channel 22) may be approximately 0.009 inch, and the thickness of the tubular wall forming body 12 may be approximately 0.0025 inch. Elongated body 12 may be produced in a variety of lengths for each needle gauge.

A distal tip 26 is fixedly coupled (e.g., laser welded) to distal end 16 of elongated body 12. As will be explained below, distal tip 26 may be comprised of a variety of materials including bio-compatible metals/alloys and bio-degradable materials. Distal tip 26 comprises a substantially solid body having a distal taper. In the illustrated embodiment, distal tip 26 comprises a non-beveled and substantially conical body (e.g., distal tip 26 may comprise a right circular cone as illustrated); however, it should be appreciated that distal tip 26 may assume other forms suitable for piercing tissue. A proximal wall 27 (FIG. 2) of distal tip 26 sealingly encloses the distal end of channel 22 to prevent injectate from exiting elongated body 12 through distal end 16. Unlike the tips of standard injection needles, distal tip 26 does not include a longitudinal bore therethrough that may clog during injection. Furthermore, distal tip 26 acts to pierce (rather than core) tissue during as injection needle 10 during insertion into tissue thus minimizing tissue trauma. Preferably, the proximal end of distal tip 26 (e.g., wall 27) has an outer diameter substantially equivalent to the outer diameter of elongated body 12 (e.g., 0.014 inch). The length and taper of distal tip 26 may be varied as desired; however, as an example, tip 26 may have a length of approximately 0.018 inch, and the outer surface of tip 26 may form an angle of approximately 21.5° with the longitudinal axis of body 12.

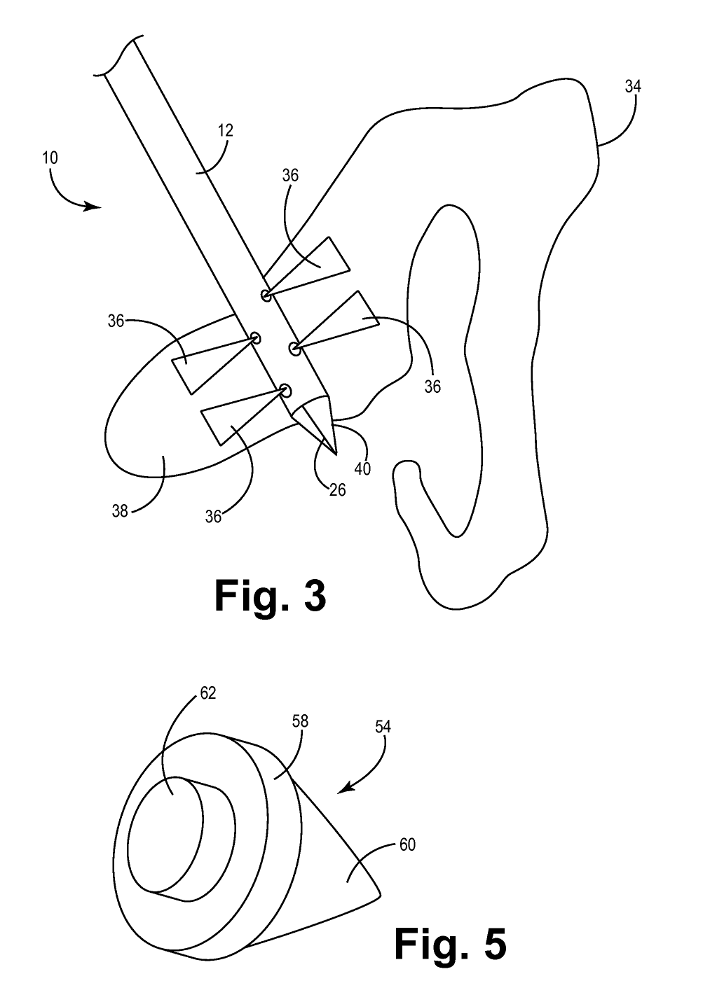

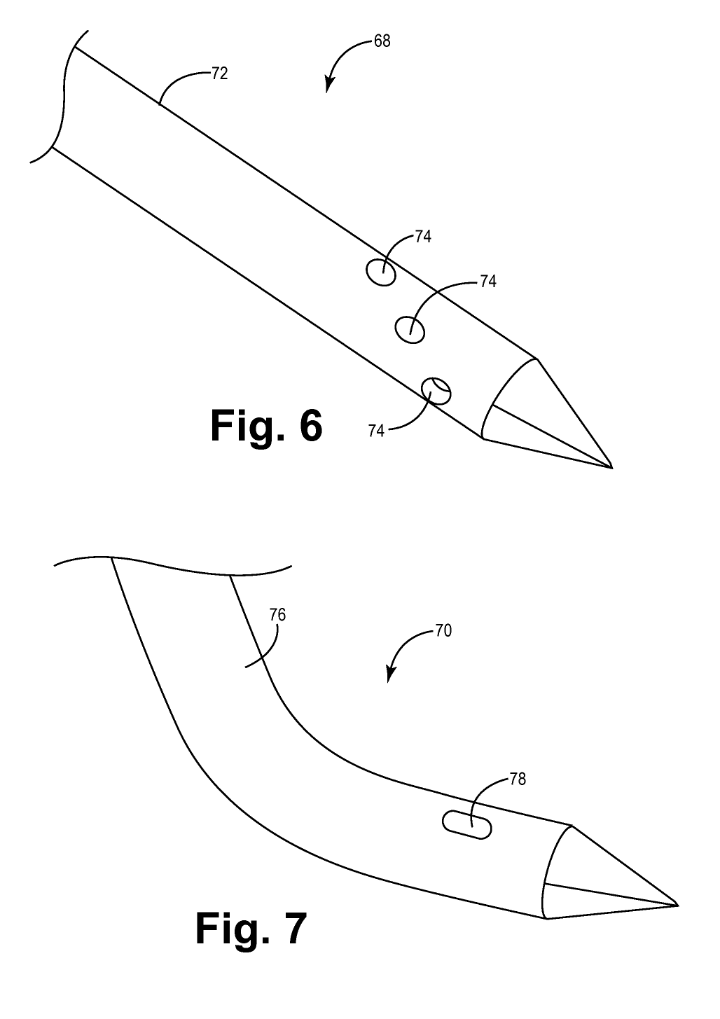

At least one lateral delivery port is provided through elongated body 12 proximate distal end 16. In the exemplary embodiment, four through holes are provided through a wall of elongated body 12. Moving distally, these through holes are numbered 28, 30, 32, and 34. The through holes are each fluidly coupled to longitudinal injectate channel 22 and permit injectate conducted thereby to exit elongated body 12. Through holes 28, 30, 32, and 34 may each comprise a pair of opposing apertures, which each extend radially from inner surface 20 to outer surface 18. The lateral delivery ports are circumferentially spaced around a distal, annular portion of elongated body 12. For example, the through holes may be arranged such that the longitudinal axis of each of through holes 28, 30, 32, and 34 is substantially orthogonal to the longitudinal axis of injectate channel 22. Furthermore, the longitudinal axes of holes 28 and 32 may be substantially perpendicular to the longitudinal axes of holes 30 and 34. Such an orthogonal arrangement provides a relatively large zone of injectate dispersal (illustrated in FIG. 3). This notwithstanding, it should be understood that a wide variety of alternative arrangements are possible, including those described below in conjunction with FIGS. 6 and 7.

The lateral delivery ports (e.g., each aperture comprising through holes 28, 30, 32, and 34) may be provided with a variety of geometries, including rectangular, oval, and/or circular cross-sections (illustrated). The cross-sectional area of the lateral delivery ports will vary depending upon application, design, and the overall dimensions of needle 10. For substantially circular delivery ports, the diameter of the lateral delivery ports may be less than 90% of the diameter of channel 16, and, in one embodiment, the diameter of the delivery ports may be substantially equivalent to 80% of the diameter of channel 16. If injection needle is to be utilized to deliver an injectate containing living cells (e.g., human cells, such as dermal fibroblasts), the dimensions of the delivery ports are preferably sufficient to maintain cell viability during injection. For example, each of the apertures comprising through holes 28, 30, 32, and 34 may have a diameter equivalent to or in excess of approximately 0.004 inch.

Each of the lateral delivery ports may have a similar or identical cross-sectional area or, in the case of circular delivery ports, a similar or identical diameter. However, in certain embodiments, it may be desirable to employ lateral delivery ports having different cross-sectional areas to encourage a substantially equal flow rate during injection and, therefore, a substantially uniform dispersal of injectate. The cross-sectional areas of the lateral delivery ports may vary in relation to the number of ports, port arrangement, port size, and the location of the ports relative to distal end 16 (or the distal end of channel 22). In the exemplary embodiment, the distance separating distal end 16 from the longitudinal axes of each through hole may be as follows: approximately 0.007 inch for through hole 28, approximately 0.013 inch for through hole 30, approximately 0.018 inch for through hole 32, and approximately 0.023 for through hole 34. The diameter of each of the apertures comprising through holes 28 and 30 may be approximately 0.004 inch, and the diameter of each of the apertures comprising through holes 32 and 34 may be approximately 0.005 inch. As alternative to varying the cross-sectional area of the lateral delivery ports, the number of lateral ports per annular section of body 12 may also increase with increasing proximity to distal tip 26.

FIG. 3 is a plan view of injection needle 10 administering a myocardial injection to atrial tissue 34. In particular, injection needle 10 is delivering an injectate 36 containing living cells (e.g., human cells, such as dermal fibroblasts) to a relatively thin atrial appendage 38. As graphically indicated in FIG. 3, the lateral ports provided through elongated body 12 are oriented such that injection needle 10 produces a relatively large, annular zone of dispersal about a distal annular portion of needle 10. As a result, a relatively large volume of atrial tissue 34 is exposed to injectate 36. Distal tip 26 has pierced through appendage 38 and thus perforated atrial tissue 34 as indicated at 40. Despite this perforation, most or all of injectate 36 is delivered into atrial tissue 34. In contrast, if injection needle 10 were a standard needle having a distal bore through tip 26, injectate 36 would be lost to the interstitial space surrounding appendage 38.

It is appropriate to note at this juncture that injection needle 10 (and other embodiments of the inventive injection needle) exhibit pressure vs. flow rate characteristics similar to those of standard injection needles. For example, injection needle 10 has shown to have an injection flow rate of approximately 10 micro-liters per second for a pressure of 27 psia (pounds per square inch absolute), which is substantially equivalent to the injection flow rate for a standard injection needle at the same pressure. Furthermore, at higher pressures (above 15 micro-liters per second), injection needle 10 has shown pressure vs. flow rate characteristics superior to those of conventional injection needles.

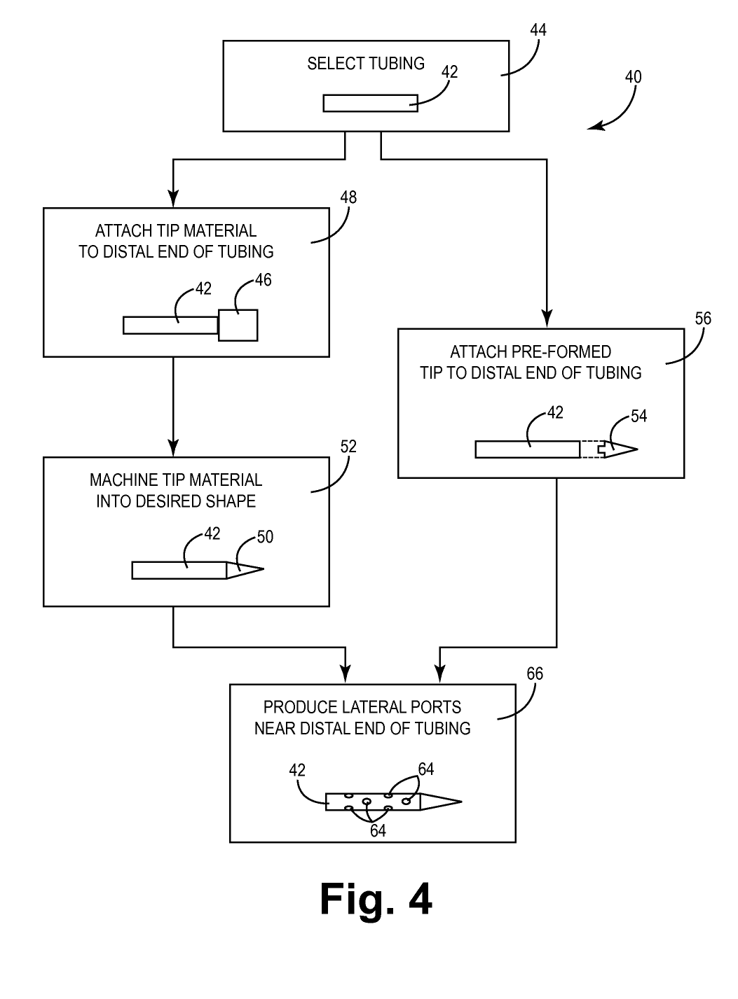

FIG. 4 is a flowchart illustrating a process 40 for producing injection needle 10 (FIGS. 1-3) and other embodiments of the inventive injection needle. Process 40 begins with the selection of tubing 42 (STEP 44) having the desired dimensions (e.g., the desired needle gauge) and comprising a suitable material. Tubing 42 may be pre-cut to a specified length or may, instead, be trimmed at a later processing stage. Tubing 42 may comprise any one of a variety of materials, including a number of bio-compatible metals (e.g., stainless steel, titanium, aluminum, etc.). However, if the produced needle is to be carried by a tissue injection catheter, it is preferable that tubing 42 is chosen to comprise a flexible, super-elastic alloy (e.g., nitinol), which may provide increased maneuverability through tortuous lumen.

After tubing 42 has been selected (STEP 44), a distal tip is fixedly attached to the distal end tubing 42. This may be accomplished in at least two manners as outlined in FIG. 4. First, a body of tip material 46 may be attached to the distal end of tubing 42 (STEP 48) by way of, for example, laser welding or soldering. The body of tip material 46 may be, for example, a segment of cylindrical wire. Tip material 46 may comprise any suitable material, including the bio-compatible metal and alloys mentioned above (e.g., nitinol). Alternatively, tip material 46 may comprise a radio-opaque material visible under fluoroscopy as described below. After attachment to the distal end of tubing 42, body of tip material 46 is machined (e.g., ground) to produce a solid tapered tip 50 (STEP 52). If grinding is utilized to shape distal tip 50, the outer diameter of the body of tip material 46 is preferably larger than that of tubing 42. If desired, chemical polishing may also be employed to form distal tip 50.

In lieu of STEPS 48 and 52, a pre-formed distal tip 54 may be attached to the distal end of tubing 42 (STEP 56). FIG. 5 is an isometric view of an exemplary pre-formed distal tip 54 including a disc-like base 58, a tapered head 60 extending distally from base 58, and a cylindrical plug portion 62 extending proximally from base 58. As described above, pre-formed distal tip 54 may comprise a variety of bio-compatible materials, including radio-opaque metals and alloys. Base 58 preferably has an outer diameter substantially equivalent to that of tubing 42. The outer diameter of plug portion 62 is preferably slightly less than the inner diameter of tubing 42; e.g., if the inner diameter of tubing 42 is 0.009 inch, the outer diameter of plug portion 62 may be approximately 0.0085 inch. The length of plug portion 62 may be, for example, 0.003 inch. To perform STEP 56, pre-formed distal tip 54 is positioned to abut the distal end of tubing 42 such that plug portion 62 extends into tubing 42, and pre-formed distal tip 54 is fixedly coupled (e.g., laser welded) to tubing 42.

The above notwithstanding, pre-formed distal tip 54 may comprise a bio-degradable material, such as polylactoglycolic acid, polyglycolic acid, polyethylene glycol, polylatic acid, polycaprolactone, or block copolymers thereof. In one embodiment, pre-formed distal tip 54 is comprised of a polymeric body impregnated with a bioactive drug or agent. In this case, distal tip 54 may be configured to detach from tubing 42 after insertion into tissue and slowly degrade to release the drug or agent in a controlled manner. Furthermore, such a distal tip 54 may also be filled with a radio-opaque material, such as barium sulfate.

After a distal tip is attached to the distal end of tubing 42 by way of STEP 56 or by way of STEPS 48 and 52, at least one lateral delivery port 64 is created through tubing 42 proximate the distal end thereof (STEP 66). For example, the lateral delivery ports may be formed by laser welding. Alternatively, electrical discharge machining may be employed wherein cutting is accomplished utilizing an electrode configured to produce a series of electric arching discharges. The electrical discharges melt and/or vaporize portions of tubing 42, which are then washed away by a dielectric fluid. To complete processing, the proximal end of tubing 42 may be trimmed to a desired length (if required), the distal tip may be sharpened, and/or the outer surface of the distal tip and the distal portion of tubing 42 may be polished.

A method has thus been provided for producing embodiments of the inventive injection needle, such as needle 10 shown in FIGS. 1-3. However, it will be appreciated by one skilled in the art that other methods may be utilized to produce the inventive injection needle or the components thereof. For example, the distal tip may be produced way of stamping from a solid needle tubing. Additionally, it should be understood that the steps employed by process 40 may be performed in any practical order; e.g., STEP 66 may be performed prior to STEP 56 or STEPS 48 and 52.

As mentioned above, the distal tip may comprise a radio-opaque material visible under fluoroscopy. Radio-opaque materials suitable for this purpose include, but are not limited to, platinum, palladium, gold, tungsten, iridium, tantalum, and rhenium. By providing a radio-opaque tip in this manner, the injection needle may be more easily guided to a target site by a flexible catheter and may more accurately administer an injection. If the distal tip comprises a radio-opaque material having a melting point higher than that of tubing 42, it may be desirable to utilize STEP 56 (as opposed to STEPS 48 and 52) to produce the injection needle; the attachment process of STEP 56 minimizes blending between the tube material and the tip material and thus helps to preserve the integrity of the image during fluoroscopy.

As stated previously, the number, arrangement, size, and shape of the lateral delivery ports may be varied as desired. To further emphasize this point, FIGS. 6 and 7 provide isometric views of two needles (i.e., needles 68 and 70) in accordance with second and third embodiments of the present invention, respectively. Referring first to needle 68 (FIG. 6), an elongated body 72 includes three through holes 74 proximate the distal end thereof. Each through hole 74 comprises two opposing circular apertures having similar cross-sectional areas. Each aperture resides at a different circumferential position around a distal annular portion of elongated body 72. More specifically, the longitudinal axis of each through hole forms a 60° angle with the longitudinal axes of the other through holes. Arrangements of this type may enlarge the zone of dispersion and may also augment the structural integrity of injection needle 68.

In contrast to needle 68 (FIG. 6), injection needle 70 (FIG. 7) comprises a curved or arched elongated body 76 having only one aperture 78 through a distal portion thereof. Aperture 78 is substantially oval, and the long axis of aperture 78 may be substantially parallel with the longitudinal axis of elongated body 76. The cross-sectional area of aperture 78 may be substantially larger than the cross-sectional areas of the apertures comprising through holes 74 (FIG. 6) or the apertures comprising through holes 28, 30, 32, and 34 (FIGS. 1-3). For example, if injection needle 56 is chosen to have a Stubs Needle Gauge of 27, the diameter of the long axis and the short axis of aperture 78 may be approximately 0.020 inch and 0.005 inch, respectively. For an injection needle having a curved or arched body (e.g., body 76 of needle 70), it may be preferable to form the body out of a super-elastic shape memory alloy, such as nitinol.

Considering the foregoing, it should be appreciated at least one embodiment of an injection needle has been provided that resists coring tissue, that provides an enlarged injectate dispersal zone, that decreases the likelihood that injectate will be lost as a result of tissue perforation, and that may be conveniently provided with a radio-opaque marker. It should further be appreciated that at least one embodiment of a method for producing such a needle has also been provided. Embodiments of the inventive needle may be utilized with a syringe, a tissue injection catheter, or any suitable needle-carrying medical device. Although the invention has been described with reference to a specific embodiment in the foregoing specification, it should be appreciated that various modifications and changes can be made without departing from the scope of the invention as set forth in the appended claims. Accordingly, the specification and figures should be regarded as illustrative rather than restrictive, and all such modifications are intended to be included within the scope of the present invention.

What is claimed is:

1. An injection needle, comprising:
an elongated body having an outer surface, an inner surface defining a longitudinal channel through the tubular body, a distal end, and at least one lateral delivery port extending from said inner surface to said outer surface proximate said distal end and in fluid communication with said longitudinal channel; and
a distal tip coupled to said distal end and comprising a radio-opaque material.

2. An injection needle according to claim 1 wherein said distal end includes an aperture therethrough in fluid communication to said longitudinal channel, and wherein said distal tip sealingly encloses said aperture.

3. An injection needle according to claim 1 wherein said elongated body is curved.

4. An injection needle according to claim 1 wherein said at least one lateral delivery port comprises a plurality of apertures spaced around a distal, annular portion of said elongated body.

5. An injection needle according to claim 1 wherein said at least one lateral delivery port has a diameter of at least approximately 0.004 inch.

6. An injectate needle according to claim 1 wherein said longitudinal channel has a first diameter and said at least one lateral delivery port has a second diameter substantially less than or equal to 90% of said first diameter.

7. An injectate needle according to claim 6 wherein said second diameter is substantially equal to 80% of said first diameter.

8. An injection needle, comprising:
a substantially tubular body including a proximal end, a distal end, an injectate channel extending from said proximal end to said distal end, and a plurality of lateral delivery ports extend radially through said substantially tubular body and circumferentially spaced around an annular portion thereof; and
a distal tip fixedly coupled to said distal end and comprising a substantially conical body.

9. An injection needle according to claim 8 wherein said substantially conical body comprises a right circular cone.

10. An injection needle according to claim 8 wherein said distal tip further comprises a plug portion extending proximally from said substantially conical body and into said longitudinal channel.

11. An injection needle according to claim 8 wherein at least a portion of said distal tip comprises a biodegradable material.

12. An injection needle according to claim 8 wherein said plurality of lateral delivery ports includes:
a first delivery port; and
a second delivery port, said second delivery port positioned closer to said distal end than is said first delivery port, and the cross-sectional area of said second delivery port being greater than the cross-sectional area of said first delivery port.

13. An injection needle according to claim 12 wherein said first delivery port includes a substantially circular cross-section having a diameter of approximately 0.0004 inch and said second delivery port includes a substantially circular cross-section having a diameter of approximately 0.0005 inch.

14. An injection needle according to claim 8 wherein said plurality of lateral delivery ports each reside at a substantially different circumferential position around said annular portion.

15. An injection needle according to claim 8 wherein said plurality of lateral delivery ports comprises a plurality of through holes orthogonally positioned with respect to the longitudinal axis of said substantially tubular body.

16. A method for producing an injection needle comprising a tubular body having at least one lateral delivery port therethrough, the method comprising:
selecting a tubing;
attaching a distal tip to the distal end of the tubing; and
producing at least one lateral port through the tubing proximate the distal tip.

17. A method according to claim 16 wherein the step of attaching a distal tip comprises:
attaching a body of tip material to the distal end of the tubing; and
machining the body of tip material into a substantially conical tip.

18. A method according to claim 16 wherein the step of attaching a distal tip comprises laser welding a pre-formed distal tip to the distal end of the tubing.

19. A method according to claim 18 wherein the pre-formed distal tip is chosen to comprise a radio-opaque material.

20. A method according to claim 16 wherein the step of producing at least one lateral port comprises electrical discharge machining.
